(19) United States
(12) Patent Application Publication
(10) Pub. No.: US 2022/0328160 A1
(43) Pub. Date: Oct. 13, 2022

(54) REHABILITATION WORK SUPPORT APPARATUS, REHABILITATION WORK SUPPORT SYSTEM, REHABILITATION WORK SUPPORT METHOD, AND COMPUTER READABLE MEDIUM

(71) Applicant: NEC Corporation, Minato-ku, Tokyo (JP)

(72) Inventors: Yuki KOSAKA, Tokyo (JP); Toshinori HOSOI, Tokyo (JP); Masahiro KUBO, Tokyo (JP); Yuan LUO, Tokyo (JP); Shigemi KITAHARA, Tokyo (JP)

(73) Assignee: NEC Corporation, Minato-ku, Tokyo (JP)

(21) Appl. No.: 17/761,676

(22) PCT Filed: Jul. 29, 2020
(86) PCT No.: PCT/JP2020/029067

§ 371 Date: Mar. 18, 2022

(30) Foreign Application Priority Data
Oct. 4, 2019 (JP) 2019-184150

(51) Int. Cl. G16H 20/30; G16H 10/60

(52) U.S. Cl. CPC G16H 20/30; G16H 10/60

(57) ABSTRACT

A rehabilitation work support apparatus, a rehabilitation work support method, and a program capable of reducing a work burden on a therapist are provided. A rehabilitation work support apparatus (1) includes a difference calculation unit (2) and a difference output unit (3). The difference calculation unit (2) calculates, for each type of ability, a difference between a target value for a patient's ability value in rehabilitation and the patient's ability value at a time before a start of the rehabilitation or during the rehabilitation. The difference output unit (3) performs control so as to output information representing the calculated difference for each type of ability.

### TECHNICAL FIELD

The present invention relates to a rehabilitation work support apparatus, a rehabilitation work support method, and a program.

### BACKGROUND ART

Patent Literature 1 discloses an information processing server that refers to facility information and information about persons to be cared for stored in a customer management database, and retrieves a rehabilitation menu in accordance with a request from a nursing facility from a rehabilitation menu database and presents the retrieved rehabilitation menu to the nursing facility side.

In recent years, there has been a need for a technology for supporting rehabilitation (e.g., a rehabilitation training or a rehabilitation therapy) (hereinafter also referred to as “rehab”) as described above, and research and development for such technology has been pursued.

It should be noted that in order to enable a patient to recover through rehabilitation, in general, it is important to set a target for a patient's ability state in advance, and a therapist such as a physical therapist should proceed with rehabilitation while being aware of the gap between this target and the current state. Further, the therapist needs to determine appropriate contents of the rehabilitation in accordance with the aforementioned gap.

### CITATION LIST

### Patent Literature

Patent Literature 1: Japanese Unexamined Patent Application Publication No. 2005-018653

### SUMMARY OF INVENTION

### Technical Problem

However, since a therapist spends a lot of time on clinical work, it is difficult for him/her to secure time to spend for work other than clinical work (e.g., checking the gap, determining contents of the rehabilitation, etc.). Therefore, there is a need to provide a technology by which a work burden on a therapist can be reduced.

Accordingly, one of the objects to be attained by example embodiments disclosed in this specification is to provide a rehabilitation work support apparatus, a rehabilitation work support method, and a program capable of reducing a work burden on a therapist.

### Solution to Problem

A rehabilitation work support apparatus according to a first aspect of the present disclosure includes:

difference calculation means for calculating, for each type of ability, a difference between a target value for a patient's ability value in rehabilitation and the patient's ability value at a time before a start of the rehabilitation or during the rehabilitation; and

difference output means for performing control so as to output information representing the calculated difference for each type of ability.

A rehabilitation work support method according to a second aspect of the present disclosure includes:

calculating, for each type of ability, a difference between a target value for a patient's ability value in rehabilitation and the patient's ability value at a time before a start of the rehabilitation or during the rehabilitation; and

performing control so as to output information representing the calculated difference for each type of ability.

A program according to a third aspect of the present disclosure causes a computer to perform:

a difference calculation step of calculating, for each type of ability, a difference between a target value for a patient's ability value in rehabilitation and the patient's ability value at a time before a start of the rehabilitation or during the rehabilitation; and

a difference output step of performing control so as to output information representing the calculated difference for each type of ability.

### Advantageous Effects of Invention

According to the present disclosure, it is possible to provide a rehabilitation work support apparatus, a rehabilitation work support method, and a program capable of reducing a work burden on a therapist.

### BRIEF DESCRIPTION OF DRAWINGS

FIG. 1 is a block diagram showing an example of a configuration of a rehabilitation work support apparatus according to an outline of an example embodiment;

FIG. 2 is a block diagram showing an example of a configuration of a rehabilitation work support system according to an example embodiment;

FIG. 3A is a schematic diagram showing an example of a screen for selecting a task (a superordinate task) that a patient desires to accomplish through rehabilitation;

FIG. 3B is a schematic diagram for explaining an example of a screen for selecting a task (a subordinate task) that a patient desires to accomplish through rehabilitation;

FIG. 3C is a schematic diagram for showing an example of a screen for selecting an exercise item (a program) for accomplishing a task;

FIG. 4 is a table showing an example of past patient information used for calculating a target value;

FIG. 5A is an example of information representing a difference for each type of ability displayed in a portable terminal device;

FIG. 5B shows an example of information representing a difference for each type of ability displayed in a non-portable terminal device;

FIG. 5C is an example of information representing an achievement level displayed in a terminal device;

FIG. 5D is an example of information representing a hospitalization progress ratio displayed in a terminal device;

FIG. 5E shows an example of information about a plurality of patients displayed in a terminal device;

FIG. 6 is a table showing past patient information used for obtaining contents of rehabilitation;

FIG. 7 is a schematic diagram showing an example of a hardware configuration of a rehabilitation work support apparatus according to an example embodiment; and

FIG. 8 is a flowchart showing an example of an outputting operation performed by a rehabilitation work support apparatus according to an example embodiment.

### DESCRIPTION OF EMBODIMENTS

### <Overview of Example Embodiment>

Prior to describing an example embodiment in detail, an outline of the example embodiment will be described.

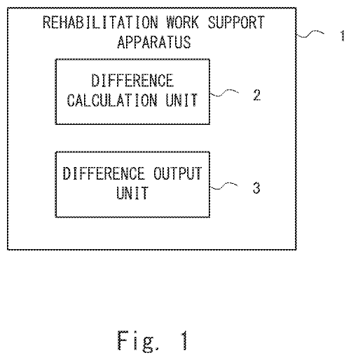

FIG. 1 is a block diagram showing an example of a configuration of a rehabilitation work support apparatus 1 according to an outline of an example embodiment. As shown in FIG. 1, the rehabilitation work support apparatus 1 includes a difference calculation unit 2 and a difference output unit 3.

The difference calculation unit 2 calculates, for each type of ability, a difference between a target value for a patient's ability value in rehabilitation (e.g., a rehabilitation training or a rehabilitation therapy) and the patient's ability value at a time before the start of the rehabilitation or during the rehabilitation.

Note that the ability value is an ability value related to patient's activities in daily life, such as ADL (Activities of Daily Living) or IADL (Instrumental Activities of Daily Living). That is, the ability value for each type of ability is an ability value for each type of patient's activity in daily life. For example, the ability value for each type of patient's activity in daily life is an ability value for eating movements, an ability value for toilet movements, or the like.

The difference output unit 3 performs control so as to output information representing the difference for each type of ability calculated by the difference output unit 2. The difference output unit 3 outputs this information to other apparatuses (e.g., a terminal device). As a result, the difference for each type of ability is displayed on a display of the other apparatus. Note that the difference output unit 3 may perform control so as to display the differences on a display provided in the rehabilitation work support apparatus 1.

According to the rehabilitation work support apparatus 1, a therapist can easily understand the gap between a target for a patient's ability and a current state thereof for each type of ability. Therefore, the therapist can reduce the time and effort required to check the gap between the target for the patient's ability and the current state thereof. That is, according to the rehabilitation work support apparatus 1, it is possible to reduce the work burden on the therapist.

<Details of Example Embodiment>

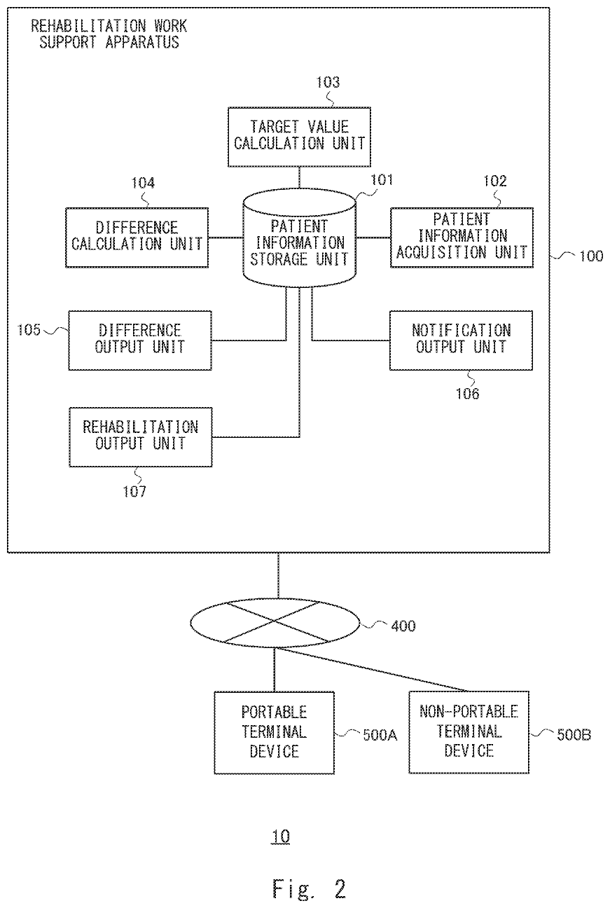

An example embodiment according to the present invention will be described hereinafter with reference to the drawings. FIG. 2 is a block diagram showing an example of a configuration of a rehabilitation work support system 10 according to an example embodiment.

The rehabilitation work support system 10 includes a rehabilitation work support apparatus 100, a portable terminal device 500A, and a non-portable terminal device 500B. In the following description, when the portable terminal device 500A and the non-portable terminal device 500B are mentioned without distinguishing them from each other, they may be referred to as terminal devices 500. The rehabilitation work support apparatus 100 is connected to the terminal devices 500 through a network 400 wirelessly or through a cable.

The rehabilitation work support apparatus 100 is configured, for example, as a server. Further, the portable terminal device 500A is an arbitrary portable terminal device such as a tablet terminal or a smartphone. Further, the non-portable terminal device 500B is a stationary terminal device such as a personal computer. The terminal device 500 is equipped with an input device and an output device, and hence is able to receive information to be transmitted to the rehabilitation work support apparatus 100 and output (display) information received from the rehabilitation work support apparatus 100.

Note that, in the example shown in FIG. 2, the rehabilitation work support system 10 includes both the portable terminal device 500A and the non-portable terminal device 500B, but it may include only one of them. Further, the numbers of portable terminal devices 500A and non-portable terminal devices 500B are not limited to the numbers shown in FIG. 2.

The rehabilitation work support apparatus 100 is an apparatus for supporting the work of a therapist who performs a rehabilitation therapy for a patient. For example, the therapist works at a certain facility such as a rehabilitation hospital and carries the portable terminal device 500A. Further, the non-portable terminal device 500B is installed, for example, in the certain facility, and the therapist can also use the non-portable terminal device 500B.

As shown in FIG. 2, the rehabilitation work support apparatus 100 includes a patient information storage unit 101, a patient information acquisition unit 102, a target value calculation unit 103, a difference calculation unit 104, a difference output unit 105, a notification output unit 106, and a rehabilitation output unit 107.

Patient information is stored in the patient information storage unit 101.

The patient information includes patient information for a target patient (target patient information) and patient information for past patients (past patient information). Note that the target patient is a patient who will start rehabilitation or is presently performing rehabilitation. The past patient is a patient who performed rehabilitation in the past.

The patient information is information about a patient. Specifically, for example, the patient information includes data items such as patient's attributes, patient's symptoms, ability value information, contents of rehabilitation performed in the past, and information about a therapist in charge. However, these data items are merely examples, and the patient information is not limited to them. Note that, in the target patient information, some of these data items may have NULL values (null values) because data in them are not determined. For example, regarding the contents of performed rehabilitation, it is a data item that will be determined after the rehabilitation is performed, so this data item has a NULL value before the rehabilitation is performed. Data in each data item in the patient information is, for example, expressed by a numerical code.

Although the rehabilitation work support apparatus 100 includes the patient information storage unit 101 in this example embodiment, the patient information storage unit 101 may be implemented by an external apparatus. In such a case, the rehabilitation work support apparatus 100 may acquire the patient information from this external apparatus.

The attributes of the patient include, specifically, an arbitrary attribute information such as the gender, the age, and the like of the patient. The ability value is an ability value related to patient's activities in daily life as described above, and is, for example, an ability value related to the ADL or the IADL.

In this example embodiment, the ability value is, specifically, an evaluation score for each evaluation item in an FIM (Function Independence Measure). The evaluation item corresponds to the type of ability. For example, in the FIM, evaluations are made for 18 different items. In this case, the patient information includes data on ability values (i.e., evaluation scores in the FIM) for the 18 types of abilities. Note that although the FIM is used as an example of ability values in this example embodiment, other ability values may also be used. Further, although the ability value is a value for each of a plurality of types of abilities (a value for each FIM item) in this example embodiment, the ability value may be an ability value for only one type of ability.

The ability value information includes patient's ability values at various times (e.g., ability values when the patient is admitted to a hospital, ability values during the hospitalization, and ability values when the patient is discharged from the hospital), target values for the patient's ability value in rehabilitation, and differences (gaps) between ability values at a certain time and their target values. Note that since the differences can be calculated from ability values at a certain time and their target values, the ability value information may include ability values at a certain time instead of including the differences. The certain time is, for example, when the patient is admitted to a hospital, one week after the admission, two weeks after the admission, three weeks after the admission, and so on. That is, the ability value information includes a difference between each of periodically-measured ability values measured in the period from the admission to the hospital to the discharge therefrom and the target value.

The contents of rehabilitation include, for example, tasks that the patient desires to accomplish through the rehabilitation and contents (programs) of practices for accomplishing the tasks. Regarding the tasks, a superordinate task(s) and a subordinate task(s) may be set.

The patient information acquisition unit 102 acquires the above-described patient information and stores the acquired patient information in the patient information storage unit 101. In this example embodiment, the patient information acquisition unit 102 provides a GUI (Graphical User Interface) to the terminal device 500 and acquires patient information entered to the terminal device 500. In particular, the patient information acquisition unit 102 specifies contents of rehabilitation performed by the patient by receiving a choice(s) selected from a plurality of choices in regard to the contents of the rehabilitation from the user (e.g., the therapist).

Here, recording of contents of commonly practiced rehabilitation will be discussed. In general, contents of rehabilitation are recorded by describing them in a free format. In such a case, even if the contents of rehabilitation sessions are the same as each other, it is difficult to manage information about them as the contents of the same rehabilitation sessions because their descriptions are different from each other. That is, it is difficult to analyze the data. In contrast, in this example embodiment, as described above, the contents of rehabilitation are specified by receiving a choice selected from a plurality of choices prepared in advance, so the data can be acquired in a format that can be easily handled in data processing.

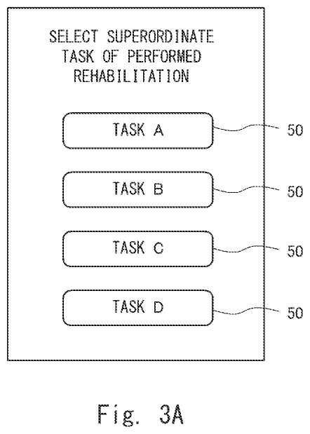

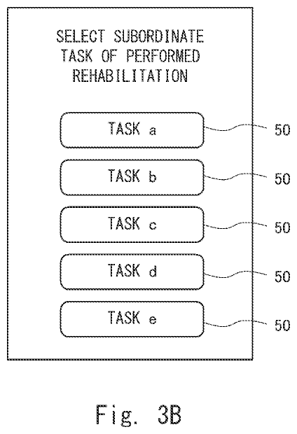

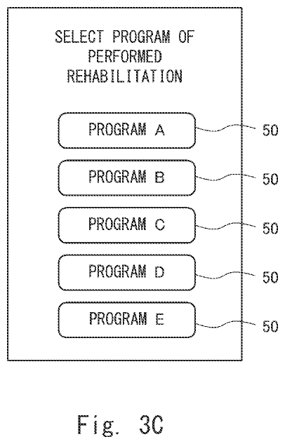

FIGS. 3A to 3C are schematic diagrams showing examples of screens (e.g., windows) that are displayed on the display of the portable terminal device 500A (e.g., the smartphone) when the patient information acquisition unit 102 acquires the contents of rehabilitation. Specifically, FIG. 3A is a schematic diagram showing an example of a screen (e.g., a window) for selecting a task (a superordinate task) that the patient desires to accomplish through the rehabilitation. FIG. 3B is a schematic diagram showing an example of a screen for selecting a task (a subordinate task) that the patient desires to accomplish through the rehabilitation. FIG. 3C is a schematic diagram showing an example of a screen for selecting contents (a program) of a practice for accomplishing a task.

The therapist selects, among the choices 50, a choice that represents the contents of the rehabilitation performed by the patient. Note that the user may be able to make a more detailed selection when he/she specify contents (a program) of a practice. For example, contents (a program) of a practice may be specified by selecting an item for each of the posture of the patient, a part of the body for which the rehabilitation is performed, and contents of an exercise. Further, other information, in addition to the contents of the rehabilitation, may also be specified by receiving a choice selected from a plurality of choices. The patient information acquisition unit 102 can also acquire patient information from the non-portable terminal device 500B, and similar screens (i.e., similar windows) may be displayed on the non-portable terminal device 500B. As described above, in this example embodiment, the patient information includes information specified as described above.

Note that the patient information acquisition unit 102 may also be referred to as a rehabilitation specification unit.

The target value calculation unit 103 calculates a target value(s) for a patient's ability value(s) in the rehabilitation. The target value calculation unit 103 calculates the target value by inputting target patient information into a prediction model using information about a plurality of patients in the past (hereinafter also referred to as a plurality of pieces of past patient information). Specifically, the target value calculation unit 103 predicts an ability value of the target patient at the time when the patient is discharged from the hospital (hereinafter also referred to as “at the time of the discharge”) by using data on ability values of past patients for each type of ability at the time of the discharge (i.e., ability values after rehabilitation), and uses the result of the prediction as a target value.

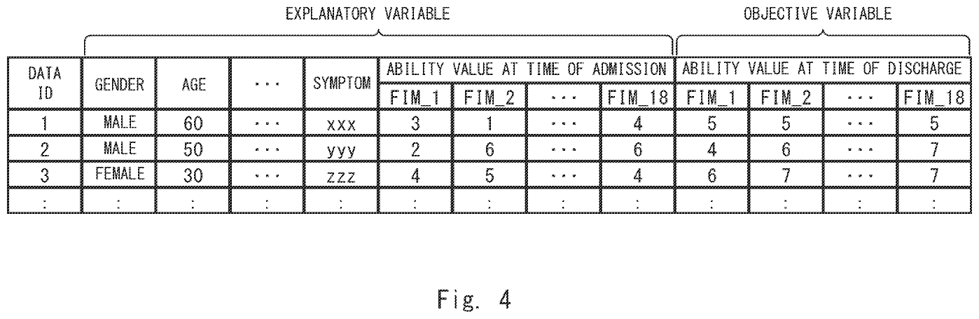

The calculation of a target value performed by the target value calculation unit 103 according to this example embodiment will be described hereinafter in detail. FIG. 4 is a table showing an example of past patient information used for the calculation of a target value. Note that, in FIG. 4, only items that used to calculate target values in this example embodiment are extracted from all the items included in the past patient information and are shown as examples.

In this example embodiment, as an example, the target value calculation unit 103 makes a prediction as follows. That is, the target value calculation unit 103 makes a prediction by using a linear regression model in which patient information representing patient's conditions at the time when the patient is admitted to the hospital (hereinafter also referred to as “at the time of the admission”) is used as explanatory variables and ability values for respective types of abilities at the time of the discharge (ability values after the rehabilitation) are used as objective variables. Note that the parameters of the linear regression model can be determined by applying a known method such as a least-squares method to the past patient information. The patient information representing the patient's conditions at the time of the admission may include at least an ability value for each type of ability at the time of the admission, and may also include the patient's conditions at the time of the admission, such as the gender, the age, and symptoms. The target value calculation unit 103 inputs information used as the explanatory variables (patient information representing the patient's conditions at the time of the admission) included in the target patient information into the linear regression model in which parameter values have already been learned, and thereby obtains the results of the predictions of the ability values after the rehabilitation. Further, the prediction results are output as target values for the target patient.

Note that, in this example embodiment, the target value calculation unit 103 makes, as an example, a prediction using a linear regression model as a prediction model. However, the prediction model is not limited to such models and may be an arbitrary machine learning model for solving a regression problem. For example, Support vector regression may be used as the prediction model. The target value calculation unit 103 stores the calculated target values as the target patient information in the patient information storage unit 101.

As described above, in this example embodiment, since the target value calculation unit 103 calculates target values for each target patient, a therapist can reduce the effort he/she needs to spend to examine the target values. Note that the target values may be determined by the therapist. Further, the target value calculation unit 103 may calculate target values as reference values that the therapist uses to determine target values.

The difference calculation unit 104 corresponds to the difference calculation unit 2 in FIG. 1, and calculates, for each type of ability, a difference (a gap) between the target value for the patient's ability value in the rehabilitation and the current patient's ability value. The difference calculation unit 104 calculates the difference by referring to the current ability value of the target patient stored in the patient information storage unit 101 (i.e., the ability value of the target patient before the start of the rehabilitation or during the rehabilitation) and the target value for the target patient. Note that, during the rehabilitation, the ability values for the target patient are checked by the therapist as appropriate, and the latest ability values are stored in the patient information storage unit 101. For example, the difference calculation unit 104 periodically calculates the differences for each target patient. The difference calculation unit 104 stores the calculated differences as target patient information in the patient information storage unit 101.

The difference output unit 105 corresponds to the difference output unit 3 shown in FIG. 1, and performs control so as to output information representing a difference for each type of ability calculated by the difference calculation unit 104 to the terminal device 500. For example, when an application running on the terminal device 500 requests such information, the difference output unit 105 refers to the patient information storage unit 101 and transmits information about differences for each patient to the terminal device 500. Specifically, for example, when a user such as a therapist provides an input for instructing to display such information to an application running on the terminal device 500, the request for the information is transmitted from the terminal device 500 to the rehabilitation work support apparatus 100. As a result, the application running on the terminal device 500 displays the information received from the rehabilitation work support apparatus 100 on the display of the terminal device 500. Note that the output (the display) of the differences may be performed without a request from the terminal device 500.

Further, when the differences are displayed, differences for a plurality of target patients may be sorted in ascending or descending order of the differences and displayed in the sorted order.

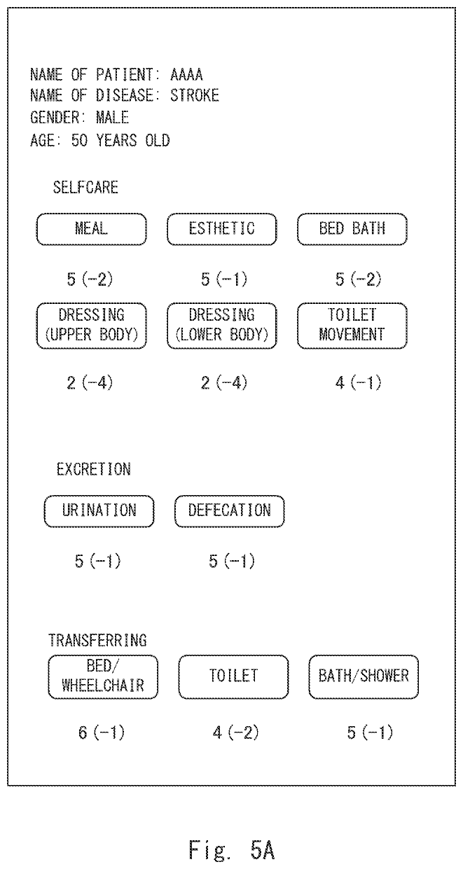

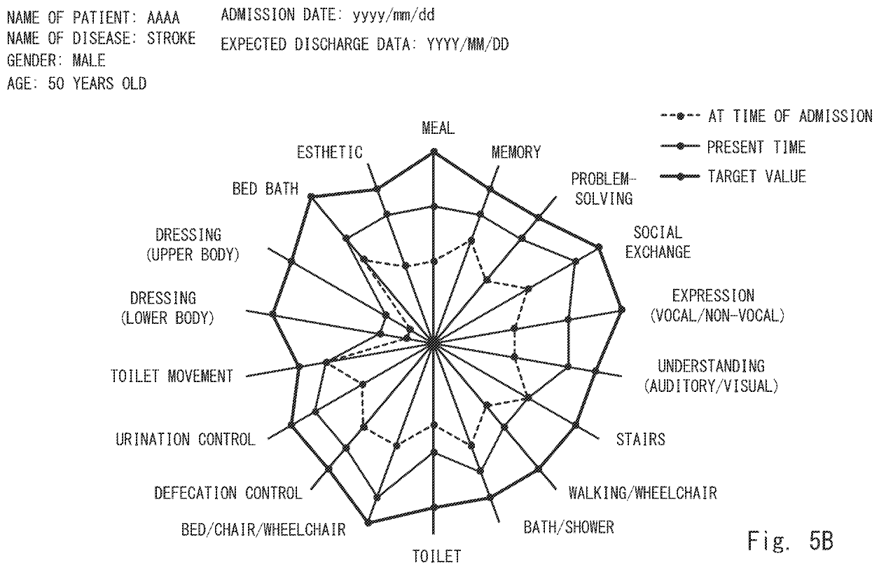

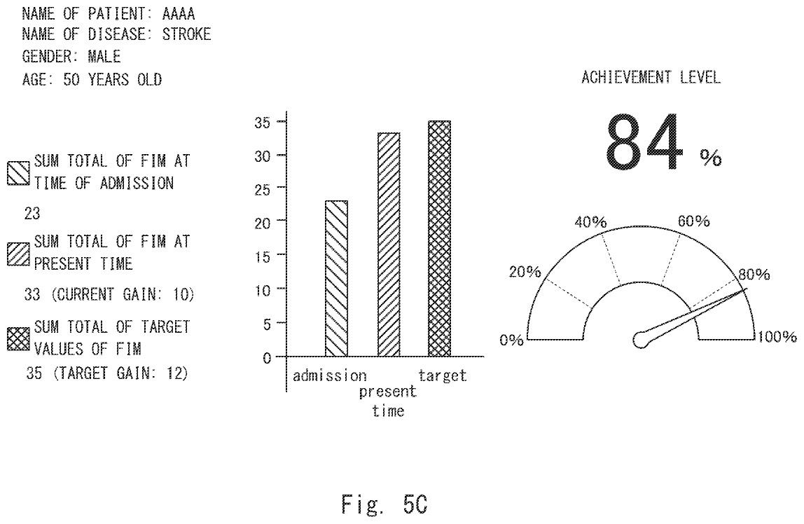

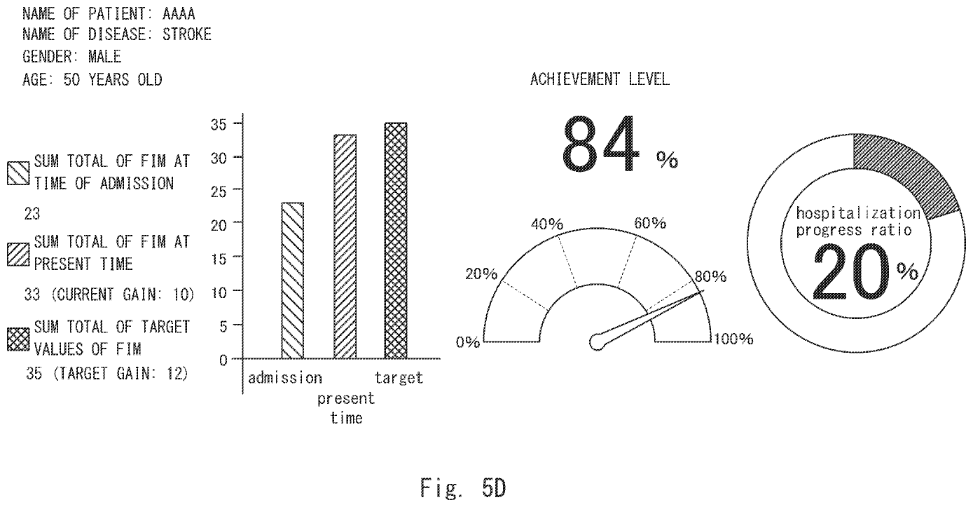

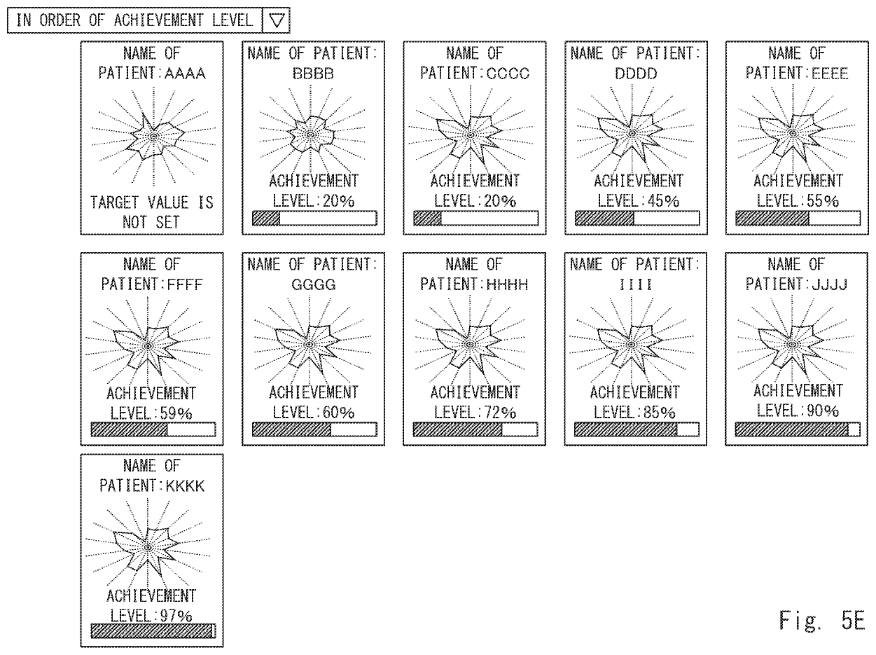

FIG. 5A is an example of displayed information representing a difference for each type of ability in the portable terminal device 500A. In the example shown in FIG. 5A, a numerical value representing a current value and that representing a difference are shown for each type of ability. Note that, in FIG. 5A, the differences are shown in parentheses. By the above-described display, a user can easily look at a list of ability values for respective types of abilities.

Further, FIG. 5B is an example of displayed information representing a difference for each type of ability in the non-portable terminal device 500B. In the example shown in FIG. 5B, a current value and a target value for each type of ability are shown by using a radar chart (a spider chart). In this way, a user can easily understand the difference between the current value and the target value for each type of ability in a visual manner. Note that a radar chart in which difference values are plotted may be displayed.

Further, a display like the one shown in FIG. 5B may be displayed on the portable terminal device 500A, and a display like the one shown in FIG. 5A may be displayed on the non-portable terminal device 500B. Further, the display of numerical values as shown in FIG. 5A and the display of a graph as shown in FIG. 5B may be used in combination. Further, as shown in FIG. 5B, ability values at the time of the admission may be displayed. Further, patient information other than the ability values may be displayed along with the information about the ability values.

Further, as shown in FIG. 5C, information representing the achievement level in the rehabilitation may be displayed on the terminal device 500. Note that the achievement level is a ratio of the gain of the current ability value to the gain of the target ability value. That is, the achievement level is expressed as “Achievement Level”=“Gain of Current Ability”/ “Gain of Target Ability”. The gain of the current ability value (specifically, the current FIM value) is a numerical value indicating the amount of recovery at the present time. Specifically, the gain of the current ability value is a value that is obtained by subtracting the sum total of the ability values (the FIM values) at the time of the admission from the sum total of the current ability values (the FIM values). Further, the gain of the target ability value (specifically, the target FIM value) is a numerical value indicating the target amount of recovery. Specifically, the gain of the target ability value is a value that is obtained by subtracting the sum total of the ability values (the FIM values) at the time of the admission from the sum total of the target values for the ability values (the FIM values). Note that the achievement level is calculated, for example, by the difference calculation unit 104, and is output to the terminal device 500 by the difference output unit 105 so that it is displayed thereon. By displaying the achievement level as described above, a user can easily understand the gap between the target value and the current value.

Further, as shown in FIG. 5D, not only the achievement level but also information representing a hospitalization progress ratio may be displayed on the terminal device 500. Note that the hospitalization progress ratio is a ratio of a hospitalization progress period to an expected hospitalization period. That is, the hospitalization progress ratio is expressed as “Hospitalization Progress Ratio” =“Hospitalization Progress Period”/ “Expected Hospitalization Period”. The hospitalization progress period is a numerical value indicating the progress time (i.e., elapsed time) from the date of the admission, and specifically, is the number of days obtained by subtracting the date of the admission from the present date. Further, the expected hospitalization period is a numerical value indicating the expected hospitalization period, and specifically, is the number of days obtained by subtracting the date of the admission from the expected discharge date. Note that the hospitalization progress ratio is, for example, calculated by the difference calculation unit 104, and is output to the terminal device 500 by the difference output unit 105 so that it is displayed thereon. By displaying the hospitalization progress ratio along with the achievement level at the same time as described above, a user can also easily understand whether or not the magnitude of the gap between the target value and the current value is appropriate.

For example, when the achievement level is 80% and the hospitalization progress ratio is 80%, it means that 80% of the expected amount of recovery has been achieved at the point when 80% of the expected hospitalization period has elapsed. Therefore, it can be easily understood that since it is necessary to accomplish only 20% of the recovery in the remaining 20% of the hospitalization period at the current pace, the recovery is going well. Further, for example, when the achievement level is 84% and the hospitalization progress ratio is 20% as in the case of the example shown in FIG. 5D, it means that 84% of the expected amount of recovery has been achieved at the point when 20% of the expected hospitalization period has elapsed. Therefore, in this case, it can be easily understood that the patient is recovering faster than expected.

Further, as shown in FIG. 5E, a list of pieces of information about a plurality of patients may be displayed on the terminal device 500. Note that, for example, as shown in FIG. 5E, the pieces of information may be sorted according to the achievement level of each patient and displayed in the sorted order. Note that although a radar chart (a spider chart) is displayed for each patient in the example shown in FIG. 5E, numerical values may be displayed as in the case of the display shown in FIG. 5A. By sorting the pieces of information according to the achievement level and displaying them in the sorted order, it is possible to find more quickly a patient whose achievement level is low and hence for whom the therapist needs to cope with.

When there is a target patient for whom the difference calculated by the difference calculation unit 104 is larger than a predetermined value (i.e., a patient whose recovery status may be behind the schedule), the notification output unit 106 performs control so as to output a notification message notifying the therapist or the like of the existence of such a target patient. In this way, it is possible to make the therapist aware of the existence of such a patient. Specifically, for example, the notification output unit 106 transmits a notification message to the terminal device 500. In the terminal device 500, the received notification message is displayed on the display. Note that, in the terminal device 500, the notification message may be output in the form of a sound (e.g., a voice). Further, for example, if there is a patient for which the difference is continuously equal to or larger than a predetermined value for a predetermined period, the notification output unit 106 may output a message for notifying the therapist of the like of the existence of such a patient to the terminal device 500.

Further, the notification output unit 106 may output a notification message at a predetermined notification timing. For example, the notification output unit 106 may perform control so as to output a notification message at a timing corresponding to a timing at which rehabilitation of a target patient for whom the difference calculated by the difference calculation unit 104 is equal to or larger than a predetermined value is performed. Specifically, for example, a notification may be provided at a predetermined time on the day on which the rehabilitation by the target patient is performed. Further, the predetermined time may be, for example, a fixed time in the morning or evening, a time immediately before the start of the rehabilitation of the patient, or a time immediately after the end of the rehabilitation of the patient. In such a case, for example, the notification output unit 106 checks the timing at which rehabilitation of each patient is performed by referring to a database in which the schedule of the rehabilitation of each patient is managed, and determines the notification timing of the notification message. Since a notification is provided according to the timing at which the rehabilitation of the target patient is performed as described above, it is possible to make the therapist aware of the difference or the like at a more appropriate timing.

Further, the notification output unit 106 may perform control so that when a predetermined operation has not been performed within a predetermined time after a notification message is output, it outputs the notification message again.

For example, when an operation for checking the difference for the target patient (specifically, for example, an operation for starting the above-mentioned application) is not performed in the terminal device 500 to which a notification message has been sent, the notification output unit 106 outputs the notification message again. In this way, it is possible to urge more reliably the therapist to check the difference.

Further, the notification that the terminal device 500 has received from the rehabilitation work support apparatus 100 may be displayed thereon at a predetermined timing, such as when the non-portable terminal device 500B is logged in or when an application running on the portable terminal device 500A is started. Further, the notification of the difference and the displaying thereof may be performed, among all the target patients, only for target patients whom the therapist is in charge of.

The rehabilitation output unit 107 performs control so as to output the contents of rehabilitation for the target patient. The rehabilitation output unit 107 makes a prediction by inputting the patient information of the target patient into a prediction model using a plurality of pieces of past patient information, and thereby obtain the contents of the rehabilitation for the target patient.

Specifically, the past patient information used in this prediction model and the input patient information (the target patient information) include at least a difference of an ability value. However, the information does not necessarily have to explicitly include the difference. That is, the information may include, instead of the difference, information necessary for calculating the difference, i.e., a target value and an ability value at a certain time. Further, the past patient information used in this prediction model further includes the contents of rehabilitation performed by past patients.

A process for acquiring the contents of rehabilitation performed in the rehabilitation output unit 107 in this example embodiment will be described hereinafter in detail.

In this example embodiment, the rehabilitation output unit 107 acquires the contents of rehabilitation suitable for the target patient by calculating a probability of the occurrence of each of the contents of the rehabilitation under the condition that feature values of the target patient has already been obtained by using a probability model. That is, the rehabilitation output unit 107 calculates a conditional probability for each content of the rehabilitation. In this example embodiment, specifically, as an example, I Bayes is used for the probability model. For example, when there is a request for a proposal for contents of rehabilitation from an application running on the terminal device 500, the rehabilitation output unit 107 calculates a conditional probability for each content of the rehabilitation for the target patient designated by a user in the terminal device 500.

The rehabilitation output unit 107 calculates a conditional probability P(Yₙ|Xᵤ) by calculating the below-shown expression for each content of the rehabilitation. Note that it is assumed that there are N types of contents of the rehabilitation in total. Note that the contents of the rehabilitation are, for example, information composed of a combination of a superordinate task(s), a subordinate task(s), and contents (a program) of a practice for accomplishing the tasks. However, the contents of the rehabilitation may include not all of these items but only some of them.

$$\begin{matrix}P ( {Y}_{n} \vert {X}_{u} ) \approx P ( {X}_{u} \vert {Y}_{n} ) P ( {Y}_{n} ) \\ = {\prod }_{i = 1}^{M} P ( {X}_{u - i} \vert {Y}_{n} ) P ( {Y}_{n} ) \\ = P ( {X}_{u - 1} \vert {Y}_{n} ) P ( {X}_{u - 2} \vert {Y}_{n} ) … P ( {X}_{u - M} \vert {Y}_{n} ) P ( {Y}_{n} )\end{matrix} \qquad [ Expression 1 ]$$

In the expression, X indicates feature values in M dimensions (where M is an integer equal to or greater than 1) of the patient, and in particular, Xᵤ indicates feature values in M dimensions (hereinafter also referred to as M-dimensional features) of a target patient u. Further, each of the M-dimensional feature values of the patient is represented by X_{_i} (1≤i≤M), and in particular, each of the M-dimensional feature values of the target patient u is represented by X_{u_i}. Further, Yₙ represents a nth (1≤n≤N) content of the rehabilitation. P(Yₙ|Xᵤ) is a conditional probability indicating a probability of the occurrence of a content Yₙ of the rehabilitation under the condition that Xᵤ has been obtained as a feature value of the patient. P(X^{u}|Yₙ) is a conditional probability indicating a probability of the occurrence of a feature Xᵤ under the condition that the content Yₙ of the rehabilitation has been obtained. P(Xu_{u_i}|Yₙ) is a conditional probability indicating a probability of the occurrence of a feature X_{u_i} under the condition that the content Yₙ of the rehabilitation has been obtained. P(Yₙ) is a probability of the occurrence of the content Yₙ, of the rehabilitation.

The rehabilitation output unit 107 acquires, for example, a feature value Xᵤ of a target patient u designated by the user (the therapist) from the patient information storage unit 101. Note that a GUI may be configured (or constructed) so that the target patient u can be designated from a screen (e.g., a window) in which differences of ability values are displayed.

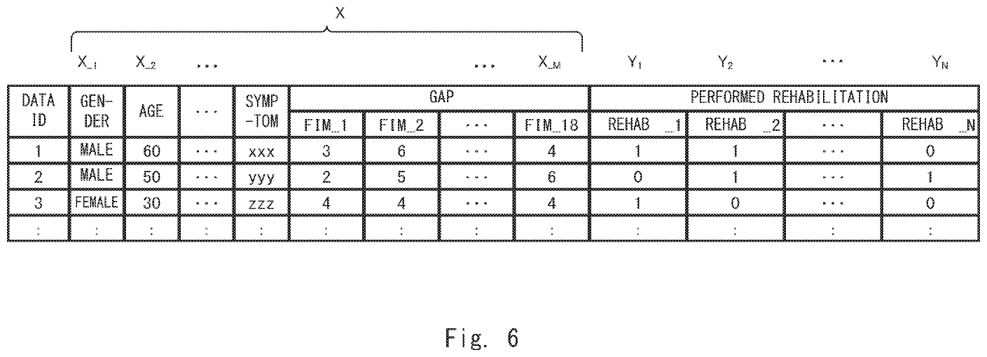

P(X_{u_i}|Yₙ) and P(Yₙ) can be calculated from statistical data of past patient information stored in the patient information storage unit 101. FIG. 6 is a table showing past patient information used for the acquisition of contents of rehabilitation. Note that, in FIG. 6, only items that used to calculate probabilities in this example embodiment are extracted from all the items included in the past patient information and are shown as examples.

In FIG. 6, the past patient information includes, as M-dimensional features, features in regard to differences (gaps) of ability values, and includes other features (e.g., features representing paient's conditions at the time of the admission, such as the gender, the age, symptoms, and ability values at the time of the admission). Note that, regarding the feature value in regard to the difference (the gap), there is a feature value for each type of ability. The feature values of the differences may be differences for all the types of abilities, or may be those for some types of abilities. Note that it is sufficient if the feature values of the differences are those for differences at a certain time. For example, they can be differences at the time of the admission or those at the time when the first week has elapsed from the admission.

Further, for each past patient, information as to which contents of the rehabilitation have been performed is recorded. In Fig“ ”, “1” is recorded for each of the contents of the rehabilitation that were already performed“a”d “0” is recorded for each of the contents of the rehabilitation that have not been performed yet. For example, in the example shown in FIG. 6, for a 50-year-old male past patient whose data ID is 2, information indicating that the second content of the rehabilitation (Rehab_2) and an Nth content of the rehabilitation (Rehab_N) were already performed is recorded.

P(X_{u_i}Yₙ) and P(Yₙ) are calculated based on such statistical data on past patient information. Therefore, it is possible to calculate a probability that a content Yₙ of the rehabilitation occurs for a target patient u having M-dimensional features Xᵤ. That is, the rehabilitation output unit 107 calculates, for each content of the rehabilitation, a probability of the occurrence of a case where a certain content of the rehabilitation is performed for a patient having certain feature values based on statistical data for past patients.

The rehabilitation output unit 107 assumes that the larger the conditional probability P(Yₙ|Xᵤ) is, the more appropriate the content of the rehabilitation is for the target patient u. Therefore, for example, the rehabilitation output unit 107 outputs, to the terminal device 500, the contents of the rehabilitation for which the calculated conditional probability is the highest or the contents of the rehabilitation for which the calculated conditional probability is higher than a predetermined threshold as the contents of rehabilitation that are recommended for the target patient u.

Note that the rehabilitation output unit 107 may output the conditional probability P(Yₙ|Xᵤ) for all the contents of the rehabilitation, or may output the conditional probability P(Yₙ|Xᵤ) for a content(s) of the rehabilitation designated by the user (the therapist). In this way, the user can understand that, for a given target patient u, which contents of the rehabilitation are appropriate and which contents thereof are inappropriate.

The rehabilitation output unit 107 may use, as the prediction model, a model that uses past patient information for past patients whose ability values after the rehabilitation meet a predetermined condition(s). That is, the above-described calculation of a probability may be performed by using only data of past patients whose ability values after the rehabilitation meet a predetermined condition(s) as the above-described statistical data. In such a case, for example, for each statistical data, a label indicating whether or not the data is data on past patients whose ability values after the rehabilitation meet a predetermined condition(s) may be added to the data so that it is possible to determine whether or not the data can be used for probability calculation.

For example, the predetermined condition may be a condition as to whether the ability value after the rehabilitation is larger than the target value. That is, the above-described calculation of a probability may be performed by using only past patient information for past patients whose ability values after the rehabilitation are larger than the target values. In such a case, it is possible to output, as the contents of the rehabilitation for the target patient, contents of the rehabilitation by which the target patient can be expected to be significantly recovered.

Further, for example, the predetermined condition may be a condition as to whether the difference between the ability value after the rehabilitation and the target value is within a predetermined range. That is, the above-described calculation of a probability may be performed by using only past patient information for past patients for whom the difference between the ability value after the rehabilitation and the target value is within a predetermined range. Note that the predetermined range is, for example, a predetermined range indicating that the difference between the ability value after the rehabilitation and the target value is small. In such a case, it is possible to output, as the contents of the rehabilitation for the target patient, contents of the rehabilitation by which the target patient can be expected to be moderately recovered.

Further, the predetermined condition may also be a condition for an ability value after the rehabilitation for a specific type of ability. For example, the contents of rehabilitation suitable for recovering a type of ability (an item in the

FIM) designated by the user (the therapist) may be output. In such a case, for example, the rehabilitation output unit 107 performs the above-described calculation of a probability by using only past patient information for past patients whose ability values after the rehabilitation for a designated ability meets a predetermined condition. Note that a GUI may be configured so that this ability can be designated from a screen (e.g., a window) in which differences of ability values are displayed.

Note that the restriction on the statistical data to be used is not limited to the above-described examples, and it is possible to restrict the statistical data based on various other aspects. For example, the statistical data may be restricted by a feature(s) (e.g., the length of service) of a therapist who performed rehabilitation therapies on past patients. That is, by using statistical data of past patients whom a therapist(s) having a desired feature(s) was in charge of, it is possible to calculate a probability for the contents of rehabilitation for the therapist(s) having the desired feature(s).

Note that, in this example embodiment, as an example, the rehabilitation output unit 107 makes a prediction by using a probability model as a prediction model. However, the prediction model is not limited to such models and may be an arbitrary machine learning model.

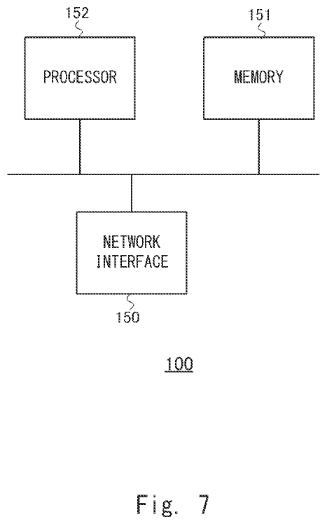

FIG. 7 is a schematic diagram showing an example of a hardware configuration of the rehabilitation work support apparatus 100. As shown in FIG.

7, the rehabilitation work support apparatus 100 includes a network interface 150, a memory 151, and a processor 152.

The network interface 150 is used to communicate with other arbitrary apparatus such as the terminal device 500.

The memory 151 is composed of, for example, a combination of a volatile memory and a non-volatile memory. The memory 151 is used to store software (a computer program) including one or more instructions executed by the processor 152, and data used for various processes performed by the rehabilitation work support apparatus 100. The patient information storage unit 101 shown in FIG. 2 is implemented, for example, by the memory 151, but may instead be implemented by other storage devices.

The processor 152 performs a process performed by each of the components shown in FIG. 2 by loading the software (the computer program) from the memory 151 and executing the loaded software. Specifically, the processor 152 performs processes performed by the patient information acquisition unit 102, the target value calculation unit 103, the difference calculation unit 104, the difference output unit 105, the notification output unit 106, and the rehabilitation output unit 107.

The processor 152 may be, for example, a microprocessor, an MPU (Micro Processor Unit), or a CPU (Central Processing Unit). The processor 152 may include a plurality of processors.

As described above, the rehabilitation work support apparatus 100 has functions as a computer. Note that, similarly, the terminal device 500 has a hardware configuration like the one shown in FIG. 7. That is, the processes performed by the terminal device 500 are implemented, for example, by having the processor execute the program.

Further, the program may be stored in various types of non-transitory computer readable media and thereby supplied to computers. The non-transitory computer readable media includes various types of tangible storage media. Examples of the non-transitory computer readable media include a magnetic recording medium (such as a flexible disk, a magnetic tape, and a hard disk drive), a magneto-optic recording medium (such as a magneto-optic disk), a CD-ROM (Read Only Memory), CD-R, CD-R/W, and a semiconductor memory (such as a mask ROM, a PROM (Programmable ROM), an EPROM (Erasable PROM), a flash ROM, and a RAM (Random Access Memory)). Further, the programs may be supplied to computers by using various types of transitory computer readable media. Examples of the transitory computer readable media include an electrical signal, an optical signal, and an electromagnetic wave. The transitory computer readable media can be used to supply programs to a computer through a wired communication line (e.g., electric wires and optical fibers) or a wireless communication line.

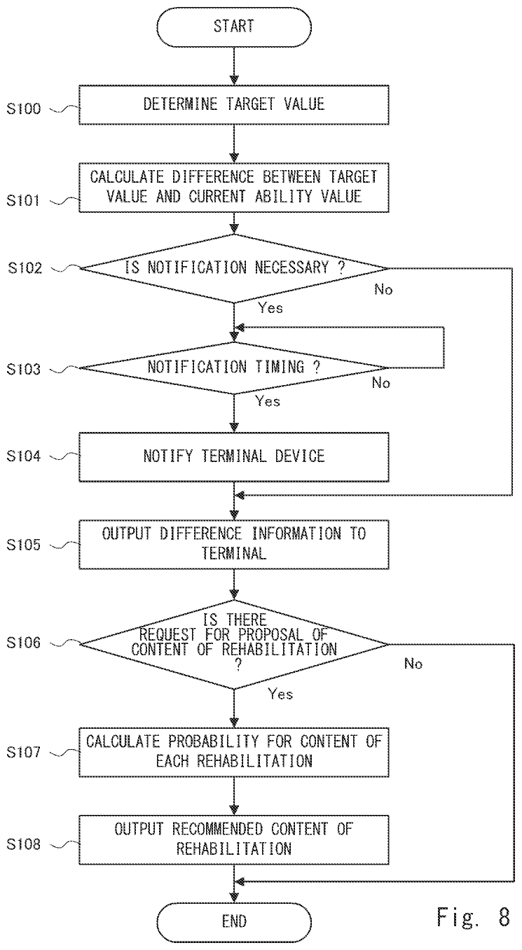

Next, a flow of an outputting operation performed by the rehabilitation work support apparatus 100 will be described. FIG. 8 is a flowchart showing an example of the outputting operation performed by the rehabilitation work support apparatus 100. The flow of the outputting operation will be described hereinafter with reference to FIG. 8.

In a step S100, a target value for a target patient is determined for each type of ability. In the step S100, the target value calculation unit 103 calculates a target value for a patient's ability value in the rehabilitation for each type of ability. The calculated target value may be determined as a target value for the target patient, or a value that is designated by a user with reference to the calculated target value may be used as a target value for the target patient.

When the target value is determined, in a step S101, the difference calculation unit 104 calculates a difference between the target value and the current patient's ability value for each type of ability, for example, at regular intervals.

Next, in a step S102, the notification output unit 106 determines whether or not a notification message needs to be output. For example, the notification output unit 106 determines whether or not there is a target patient for whom the difference calculated in the step S101 is equal to or larger than a predetermined value. When the notification is necessary (Yes at Step S102), the process proceeds to a step S103. On the other hand, when the notification is unnecessary (No at Step S102), processes in the below-described steps S103 and S104 are skipped.

In the step S103, the notification output unit 106 determines whether or not it becomes a predetermined notification timing. For example, the notification output unit 106 determines whether or not it becomes a predetermined time immediately before the start of rehabilitation for the patient for whom the notification is to be sent. When it becomes the predetermined notification timing, the process proceeds to a step S104.

In the step S104, the notification output unit 106 transmits the notification message to the terminal device 500.

When the user operates the terminal device 500, in a step S105, the difference output unit 105 transmits information about differences for each patient to the terminal device 500. That is, when the user inputs an instruction for displaying the information to an application running on the terminal device 500, the difference output unit 105 transmits the information about the differences to the terminal device 500. As a result, the differences are displayed on the terminal device 500. For example, the therapist may check the displayed differences, or/and the target patient may check them. By having the patient check the differences, it is expected that his/her motivation for the rehabilitation will improve.

Note that although a series of operations in which the process in the step S105 is performed after the processes in the steps S102 to S104 is shown in the flowchart shown in the figure, the process in the step S105 can be performed at an arbitrary timing after the step S101.

In a step S106, the rehabilitation output unit 107 determines whether or not it has received a request for a proposal for contents of the rehabilitation from the terminal device 500. When there is a request for a proposal for contents of the rehabilitation (Yes at Step S106), the process proceeds to a step S107. On the other hand, when there is no such request (No at Step S106), processes in the below-described steps S107 and S108 are skipped.

In the step S107, the rehabilitation output unit 107 calculates a probability for each content of the rehabilitation for the designated target patient.

Next, in the step S108, the rehabilitation output unit 107 outputs the contents of the rehabilitation that are recommended for the target patient to the terminal device 500 based on the result of the calculation of the probability.

Note that although a series of operations in which the process in the rehabilitation output unit 107 is performed after the process in the step S105 is shown in the flowchart shown in the figure, the process in the rehabilitation output unit 107 can be performed at an arbitrary timing.

The rehabilitation work support system 10 according to the example embodiment has been described above. According to this system, the therapist can easily understand a gap between a target for a patient's ability and a current state thereof for each type of ability. Further, the therapist can easily understand the contents of rehabilitation suitable for the patient. Therefore, according to the rehabilitation work support system 10, it is possible to reduce the work burden on the therapist.

Note that the present invention is not limited to the above-described example embodiments and various modifications can be made within the scope and spirit of the invention.

Further, the whole or part of the example embodiments disclosed above can be described as, but not limited to, the following supplementary notes.

### (Supplementary Note 1)

A rehabilitation work support apparatus comprising:

difference calculation means for calculating, for each type of ability, a difference between a target value for a patient's ability value in rehabilitation and the patient's ability value at a time before a start of the rehabilitation or during the rehabilitation; and

difference output means for performing control so as to output information representing the calculated difference for each type of ability.

### (Supplementary Note 2)

The rehabilitation work support apparatus described in Supplementary note 1, further comprising rehabilitation output means for performing control so as to predict a content of the rehabilitation for a target patient by inputting first target patient information into a first prediction model using a plurality of pieces of first past patient information, and thereby to output the content of the rehabilitation, the first target patient information being information about the target patient, and each of the plurality of pieces of first past patient information being information about a respective one of a plurality of past patients who performed rehabilitation in a past, wherein

each of the pieces of first past patient information and the first target patient information is information including at least the difference, and

each of the pieces of first past patient information is information including a content of the rehabilitation performed by a respective one of the past patients.

### (Supplementary Note 3)

The rehabilitation work support apparatus described in Supplementary note 2, wherein the first prediction model is a model using the first past patient information about the past patients whose ability values after the rehabilitation meet a predetermined condition.

(Supplementary note 4)

The rehabilitation work support apparatus described in Supplementary note 3, wherein the first prediction model is a model using the first past patient information about the past patients whose ability values after the rehabilitation are larger than the target value.

### (Supplementary Note 5)

The rehabilitation work support apparatus described in Supplementary note 3, wherein the first prediction model is a model using the first past patient information about the past patients for whom differences between the ability values after the rehabilitation and the target value are within a predetermined range.

### (Supplementary Note 6)

The rehabilitation work support apparatus described in any one of Supplementary notes 2 to 5, further comprising rehabilitation specification means for specifying a content of rehabilitation performed by a patient by receiving a choice selected from a plurality of choices in regard to the content of the rehabilitation, wherein

the first past patient information includes the content of the rehabilitation specified by the rehabilitation specification means.

### (Supplementary Note 7)

The rehabilitation work support apparatus described in any one of Supplementary notes 1 to 6, further comprising a target value calculation means for calculating the target value by inputting second target patient information into a second prediction model using a plurality of pieces of second past patient information, the second target patient information being information about a target patient, and each of the plurality of pieces of second past patient information being information about a respective one of a plurality of past patients who performed rehabilitation in a past, wherein

each of the pieces of second past patient information is information including at least, for each type of ability, an ability value of a respective one of the plurality of past patients after the rehabilitation.

### (Supplementary Note 8)

The rehabilitation work support apparatus described in any one of Supplementary notes 1 to 7, further comprising notification output means for performing control so as to output a notification message when there is a target patient for whom the difference calculated by the difference calculation means is equal to or larger than a predetermined value.

### (Supplementary Note 9)

The rehabilitation work support apparatus described in Supplementary note 8, wherein the notification output means performs control so as to output the notification message at a timing corresponding to a timing at which the rehabilitation of the target patient for whom the difference calculated by the difference calculation means is equal to or larger than a predetermined value is performed.

### (Supplementary Note 10)

The rehabilitation work support apparatus described in Supplementary note 8 or 9, wherein the notification output means performs control so as to output the notification message again when a predetermined operation has not been performed within a predetermined time after outputting the notification message.

### (Supplementary Note 11)

A rehabilitation work support system comprising: a rehabilitation work support apparatus; and a terminal device, wherein

the rehabilitation work support apparatus comprising:

difference calculation means for calculating, for each type of ability, a difference between a target value for a patient's ability value in rehabilitation and the patient's ability value at a time before a start of the rehabilitation or during the rehabilitation; and

difference output means for performing control so as to output information representing the calculated difference for each type of ability to the terminal device.

### (Supplementary Note 12)

The rehabilitation work support system described in Supplementary note 11, wherein

the rehabilitation work support apparatus further comprises rehabilitation output means for performing control so as to predict a content of the rehabilitation for a target patient by inputting first target patient information into a first prediction model using a plurality of pieces of first past patient information, and thereby to output the content of the rehabilitation, the first target patient information being information about the target patient, and each of the plurality of pieces of first past patient information being information about a respective one of a plurality of past patients who performed rehabilitation in a past,

each of the pieces of first past patient information and the first target patient information is information including at least the difference, and

each of the pieces of first past patient information is information including a content of the rehabilitation performed by a respective one of the past patients.

### (Supplementary Note 13)

A rehabilitation work support method comprising:

calculating, for each type of ability, a difference between a target value for a patient's ability value in rehabilitation and the patient's ability value at a time before a start of the rehabilitation or during the rehabilitation; and

performing control so as to output information representing the calculated difference for each type of ability.

### (Supplementary Note 14)

A non-transitory computer readable medium storing a program for causing a computer to perform:

a difference calculation step of calculating, for each type of ability, a difference between a target value for a patient's ability value in rehabilitation and the patient's ability value at a time before a start of the rehabilitation or during the rehabilitation; and

a difference output step of performing control so as to output information representing the calculated difference for each type of ability.

Although the present invention is described above with reference to example embodiments, the present invention is not limited to the above-described example embodiments. Various modifications that can be understood by those skilled in the art can be made to the configuration and details of the present invention within the scope of the invention.

This application is based upon and claims the benefit of priority from Japanese patent application No. 2019-184150, filed on Oct. 4, 2019, the disclosure of which is incorporated herein in its entirety by reference.

### INDUSTRIAL APPLICABILITY

1 REHABILITATION WORK SUPPORT APPARATUS

2 DIFFERENCE CALCULATION UNIT

3 DIFFERENCE OUTPUT UNIT

10 REHABILITATION WORK SUPPORT SYSTEM

50 CHOICE

100 REHABILITATION WORK SUPPORT APPARATUS

101 PATIENT INFORMATION STORAGE UNIT

102 PATIENT INFORMATION ACQUISITION UNIT

103 TARGET VALUE CALCULATION UNIT

104 DIFFERENCE CALCULATION UNIT

105 DIFFERENCE OUTPUT UNIT

106 NOTIFICATION OUTPUT UNIT

107 REHABILITATION OUTPUT UNIT

150 NETWORK INTERFACE

151 MEMORY

152 PROCESSOR

400 NETWORK

500A PORTABLE TERMINAL DEVICE

500B NON-PORTABLE TERMINAL DEVICE

What is claimed is:

1. A rehabilitation work support apparatus comprising:
at least one memory storing program instructions; and
at least one processor configured to execute the instructions stored in the memory to:
calculate, for each type of ability, a difference between a target value for a patient's ability value in rehabilitation and the patient's ability value at a time before a start of the rehabilitation or during the rehabilitation; and
perform control so as to output information representing the calculated difference for each type of ability.

2. The rehabilitation work support apparatus according to claim 1, wherein
the processor is further configured to execute the instructions to perform control so as to predict a content of the rehabilitation for a target patient by inputting first target patient information into a first prediction model using a plurality of pieces of first past patient information, and thereby to output the content of the rehabilitation, the first target patient information being information about the target patient, and each of the plurality of pieces of first past patient information being information about a respective one of a plurality of past patients who performed rehabilitation in a past,
each of the pieces of first past patient information and the first target patient information is information including at least the difference, and
each of the pieces of first past patient information is information including a content of the rehabilitation performed by a respective one of the past patients.

3. The rehabilitation work support apparatus according to claim 2, wherein the first prediction model is a model using the first past patient information about the past patients whose ability values after the rehabilitation meet a predetermined condition.

4. The rehabilitation work support apparatus according to claim 3, wherein the first prediction model is a model using the first past patient information about the past patients whose ability values after the rehabilitation are larger than the target value.

5. The rehabilitation work support apparatus according to claim 3, wherein the first prediction model is a model using the first past patient information about the past patients for whom differences between the ability values after the rehabilitation and the target value are within a predetermined range.

6. The rehabilitation work support apparatus according to claim 2, wherein
the processor is further configured to execute the instructions to specify a content of rehabilitation performed by a patient by receiving a choice selected from a plurality of choices in regard to the content of the rehabilitation, and
the first past patient information includes the content of the specified rehabilitation.

7. The rehabilitation work support apparatus according to claim 1, wherein
the processor is further configured to execute the instructions to calculate the target value by inputting second target patient information into a second prediction model using a plurality of pieces of second past patient information, the second target patient information being information about a target patient, and each of the plurality of pieces of second past patient information being information about a respective one of a plurality of past patients who performed rehabilitation in a past, and
each of the pieces of second past patient information is information including at least, for each type of ability, an ability value of a respective one of the plurality of past patients after the rehabilitation.

8. The rehabilitation work support apparatus according claim 1, wherein
the processor is further configured to execute the instructions to perform control so as to output a notification message when there is a target patient for whom the calculated difference is equal to or larger than a predetermined value.

9. The rehabilitation work support apparatus according to claim 8, wherein the processor is further configured to execute the instructions to perform control so as to output the notification message at a timing corresponding to a timing at which the rehabilitation of the target patient for whom the calculated difference is equal to or larger than a predetermined value is performed.

10. The rehabilitation work support apparatus according to, claim 8, wherein the processor is further configured to execute the instructions to perform control so as to output the notification message again when a predetermined operation has not been performed within a predetermined time after outputting the notification message.

11.-12. (canceled)

13. A rehabilitation work support method comprising:
calculating, for each type of ability, a difference between a target value for a patient's ability value in rehabilitation and the patient's ability value at a time before a start of the rehabilitation or during the rehabilitation; and
performing control so as to output information representing the calculated difference for each type of ability.

14. A non-transitory computer readable medium storing a program for causing a computer to perform:
a difference calculation step of calculating, for each type of ability, a difference between a target value for a patient's ability value in rehabilitation and the patient's ability value at a time before a start of the rehabilitation or during the rehabilitation; and
a difference output step of performing control so as to output information representing the calculated difference for each type of ability.
